(19) United States
(12) Patent Application Publication
(10) Pub. No.: US 2026/0182619 A1
(43) Pub. Date: Jul. 2, 2026

(54) ORAL POUCH PRODUCT AND METHOD OF MANUFACTURING THE SAME

(71) Applicant: KT&G CORPORATION, Daejeon (KR)

(72) Inventor: Sungje CHA, Daejeon (KR)

(73) Assignee: KT&G CORPORATION, Daejeon (KR)

(21) Appl. No.: 19/347,850

(22) Filed: Oct. 2, 2025

(30) Foreign Application Priority Data
Dec. 31, 2024 (KR) 10-2024-0201743

(51) Int. Cl. A24B 15/24; A24B 15/18; A24B 15/28; A24B 15/32; A24F 23/02

(52) U.S. Cl. CPC A24B 15/243; A24B 15/186; A24B 15/285; A24B 15/32; A24F 23/02

(57) ABSTRACT

An oral pouch product according to an embodiment may include a packaging material having at least part of a peripheral portion sealed to define a cavity therein, a filling material enclosed in the cavity and including an active ingredient, and a coating material applied to at least a portion of an outer surface of the packaging material and including sugar alcohol.

### CROSS-REFERENCE TO RELATED APPLICATION

This application is based on and claims priority under 35 U.S.C. § 119 to Korean Patent Application No. 10-2024-0201743, filed on Dec. 31, 2024, in the Korean Intellectual Property Office, the disclosure of which is incorporated by reference herein in its entirety.

### BACKGROUND

### 1. Field

Embodiments relate to an oral pouch product and a method of manufacturing the same. More specifically, the embodiments relate to an oral pouch product that allows a user to perceive a usage time via texture felt on a surface of the pouch product and a method of manufacturing the same.

### 2. Description of the Related Art

An oral pouch product is a type of smoking product that may be used by a user by placing it between their cheek and gum or between their gum and lip and holding it there for a certain period of time.

The oral pouch product generally includes a filling material including an active ingredient and a packaging material enclosing the filling material. The packaging material is saliva-permeable, and the active ingredient included in the enclosed filling material may be eluted by the user's saliva and provide satisfaction to the user via microvessels of their oral mucosa.

Oral pouch products differ in factors such as the overall weight or the amount of the active ingredient included in the filling material, depending on a release target of the active ingredient. Therefore, although a recommended usage time is presented depending on the release target, users face the difficulty of checking it each time.

In addition, oral pouch products are problematic because the usage end time cannot be confirmed and the longer they remain in the mouth beyond the recommended usage time, the more likely it is to cause a foreign-body sensation in the throat, and the active ingredient may be absorbed via the digestive system, causing gastrointestinal discomfort or nausea.

### SUMMARY

To solve the above problems of oral pouch products, embodiments provide an oral pouch product that allows users to perceive a usage time via texture felt on a surface of the pouch product during use.

Problems to be solved through embodiments of the disclosure are not limited to the above-described problems, and problems not mentioned may be clearly understood by one of ordinary skill in the art to which the embodiments belong from the description and accompanying drawings.

Additional aspects will be set forth in part in the description which follows and, in part, will be apparent from the description, or may be learned by practice of the presented embodiments of the disclosure.

An oral pouch product according to an embodiment may include a packaging material having at least part of a peripheral portion sealed to define a cavity therein, a filling material enclosed in the cavity and including an active ingredient, and a coating material applied to at least a portion of an outer surface of the packaging material and including sugar alcohol.

A method of manufacturing an oral pouch product, according to an embodiment, may include a) providing a filling material including an active ingredient, and b) sealing at least part of a peripheral portion of a packaging material and enclosing the filling material in an internal cavity of the packaging material, and may further include c) before or after operation b), applying a coating material including sugar alcohol to at least a portion of an outer surface of the packaging material.

### BRIEF DESCRIPTION OF THE DRAWINGS

The above and other aspects, features, and advantages of certain embodiments

of the disclosure will be more apparent from the following description taken in conjunction with the accompanying drawings, in which:

FIG. 1 is a diagram schematically illustrating an oral pouch product according to an embodiment; and

FIGS. 2A and 2B are planar cross-sectional views of an oral pouch product according to embodiments, taken along a line A-A′ of FIG. 1.

### DETAILED DESCRIPTION

Regarding the terms in the various embodiments, the general terms which are currently and widely used are selected in consideration of functions of structural elements in the various embodiments of the present disclosure. However, meanings of the terms can be changed according to intention, a judicial precedence, the appearance of a new technology, and the like. In addition, in certain cases, terms which can be arbitrarily selected by the applicant in particular cases. In such a case, the meaning of the terms will be described in detail at the corresponding portion in the description of the present disclosure. Therefore, the terms used in the various embodiments of the present disclosure should be defined based on the meanings of the terms and the descriptions provided herein.

In addition, unless explicitly described to the contrary, the word “comprise” and variations such as “comprises” or “comprising” will be understood to imply the inclusion of stated elements but not the exclusion of any other elements.

As used herein, hen an expression such as “at least any one” precedes arranged elements, it modifies all elements rather than each arranged element. For example, the expression “at least any one of a, b, and c” should be construed to include a, b, c, or a and b, a and c, b and c, or a, b, and c.

Hereinafter, the present disclosure will now be described more fully with reference to the accompanying drawings, in which exemplary embodiments of the present disclosure are shown such that one of ordinary skill in the art may easily work the present disclosure. However, the present disclosure may be implemented in various different forms, and is not limited to the embodiments described herein.

Throughout the specification, embodiments are arbitrary divisions made to facilitate the explanation of the invention, and need not be mutually exclusive. For example, components disclosed in one embodiment may be applied to and/or implemented in other embodiments and may be modified and/or implemented within the scope of the disclosure.

In addition, the terms used in the disclosure are for describing embodiments and are not intended to limit the embodiments. Unless specifically stated otherwise, singular terms in the disclosure also include the plural.

The term “oral pouch product” used throughout the specification may refer to an active ingredient-containing filling material for oral use and a saliva-permeable pouch packaging the same. The oral pouch product may be used by a user placing the oral pouch product between their cheek and gum, or between their gum or lip, and maintaining it for a certain period of time. When the oral pouch product is used in the user's oral cavity, the filling material includes an active ingredient that is released by saliva without a crushing action such as mastication.

Hereinafter, the disclosure will be described in detail with reference to the accompanying drawings, in which embodiments of the disclosure are shown such that those of ordinary skill in the art may easily work the disclosure. The disclosure is not limited to the embodiments described herein.

Some components shown in the drawings may be illustrated with exaggerated dimensions or proportions. In addition, components shown in one drawing may not be shown in other drawings.

Hereinafter, embodiments will be described in detail with reference to the drawings.

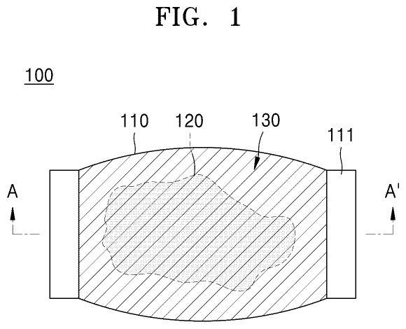

FIG. 1 is a diagram schematically illustrating an oral pouch product according to an embodiment.

According to an embodiment, an oral pouch product 100 has a size and structure that may comfortably fit between a user's cheek and gum or between their gum and lip. The oral pouch product 100 may be formed to have various shapes, including spherical, quadrangular, rectangular, crescent, oval, and cubic shapes, and may be formed to have various sizes. For example, although FIG. 1 illustrates that the oral pouch product 100 has a rectangular shape, the disclosure is not limited thereto.

According to an embodiment, the oral pouch product 100 may include a packaging material 110, a filling material 120, and a coating material 130. According to an embodiment, the oral pouch product 100 including the filling material 120, the packaging material 110, and the coating material 130 may have a total weight of 0.2 g to 1.5 g.

The oral pouch product 100 according to an embodiment may include the packaging material 110 having at least part of a peripheral portion 111 sealed to define a cavity therein, the filling material 120 that is enclosed in the cavity and includes an active ingredient, and the coating material 130 applied to at least a portion of an outer surface of the packaging material 110 and including sugar alcohol.

For example, as shown in FIG. 1, the peripheral portion 111 of the packaging material 110, where the packaging material 110 is sealed, may be both end portions of the packaging material 110, but the disclosure is not limited thereto.

The packaging material 110 for the oral pouch product 100 may be made of a material that has appropriate saliva permeability and possesses a certain strength so as not to dissolve in saliva and not to tear. In addition, the packaging material 110 for the oral pouch product 100 may be made of a material that has a level of softness suitable for use inside the oral cavity of the user. Furthermore, the packaging material 110 for the oral pouch product 100 may be a material that has sealability, so as to include the filling material 120 in its inner space. For example, the packaging material 110 may be made of a nonwoven fabric material.

The packaging material 110 of the oral pouch product 100 according to an embodiment may have at least part of the peripheral portion 111 sealed to define a cavity therein, and the filling material 120 may be enclosed in the cavity of the packaging material 110. For example, the peripheral portion 111 may be sealed by heat welding or ultrasonic welding.

The packaging material 110 according to an embodiment may be a nonwoven fabric made of fibers having a fineness of 1 denier or more or a diameter of 10 μm or more. As one example, the packaging material 110 may be a nonwoven fabric made of staple fibers having a fineness of about 2 denier to about 10 denier or a diameter of about 10 μm to about 30 μm. As another example, the packaging material 110 may be a nonwoven fabric made of staple fibers having a fineness of about 3 denier to about 7 denier or a diameter of about 15 μm to about 25 μm. Staple fibers are fibers having a discontinuous length and may be obtained by cutting continuous filaments.

The packaging material 110 according to an embodiment may have a basis weight of about 10 g/m² to about 50 g/m². Basis weight refers to the weight per unit area, and when the basis weight of the packaging material 110 is greater than the above range, moisture transfer properties may be reduced, whereas when the basis weight of the packaging material 110 is less than the above range, the strength of a pouch may be reduced.

The packaging material 110 according to an embodiment may have a thickness of about 50 μm to about 300 μm. When the thickness of the packaging material 110 is greater than the above range, moisture transfer properties may be reduced, whereas when the thickness of the packaging material 110 is less than the above range, the strength of the pouch may be reduced, causing the pouch to rupture easily.

In addition, the filling material 120 for the oral pouch product 100 may include an active ingredient. The active ingredient may be an ingredient that dissolves upon contact with saliva. For example, the active ingredient may be nicotine. The amount of nicotine may be determined according to a desired strength of a nicotine pouch. As an example, the filling material 120 may include nicotine-containing granules.

Nicotine may be included in the form of nicotine base and/or nicotine salt. For example, the nicotine salt may be one or more selected from the group consisting of nicotine hydrochloride, nicotine dihydrochloride, nicotine monotartrate, nicotine bitartartrate, nicotine bitartartrate dihydrate, nicotine sulfate, nicotine zinc chloride monohydrate, nicotine salicylate, nicotine benzoate, and nicotine polacrilex.

The filling material 120 of the oral pouch product 100 according to an embodiment may include one or more selected from the group consisting of an active ingredient, a binder, a pH regulator, and a flavoring agent.

According to an embodiment, the binder that may be included in the filling material 120 may prevent decomposition of the active ingredient during storage, and may provide a desired release rate of the active ingredient. For example, the binder may include at least one selected from the group consisting of hydroxypropyl cellulose (HPC), low-substituted hydroxypropyl cellulose (L-HPC), hydropropyl methylcellulose (HPMC), methyl cellulose (MC), polyvinyl pyrrolidone (PVP), and carboxymethyl cellulose (CMC).

According to an embodiment, the pH regulator, which may be included in the filling material 120, may adjust the pH of a nicotine pouch filler to a level at which nicotine may be absorbed, such that when the nicotine pouch is placed in the oral cavity, nicotine may be converted into a form of free-based nicotine for rapid absorption. For example, the pH regulator may be at least one selected from the group consisting of potassium hydroxide (KOH), sodium hydroxide (NaOH), potassium carbonate (K₂CO₃), sodium carbonate (Na₂CO₃), potassium bicarbonate (KHCO₃), and sodium bicarbonate (NaHCO₃), but the disclosure is not limited thereto.

According to an embodiment, the flavoring agent that may be included in the filling material 120 may include a plant-derived flavoring agent such as menthol, cinnamon, sage, herbs, chamomile, licorice, lavender, bergamot, lemon, orange, jasmine, ginger, vanilla, spearmint, peppermint, acacia, coffee, celery, sandalwood, or cocoa. The flavoring agent is added to satisfy a smoker's taste, and is not limited to the above-described flavoring agent.

The oral pouch product 100 according to an embodiment may be in a dry or wet form. This may be determined according to the amount of moisture in the filling material 120. The filling material 120 may include one or more of a dry mixture and a wet mixture. The dry mixture may include 3 wt % or less of moisture based on the total weight of the filling material 120. The wet mixture may include 40 wt % or more of moisture based on the total weight of the filling material 120.

The coating material 130 for the oral pouch product 100 according to an embodiment may include sugar alcohol and may be applied to at least a portion of the outer surface of the packaging material 110. For example, the sugar alcohol may be one or more selected from the group consisting of isomaltol, erythritol, xylitol, sorbitol, maltitol, and mannitol.

According to an embodiment, the coating material 130 may be formed by spinning a coating solution including sugar alcohol onto at least a portion of the outer surface of the packaging material 110. The coating solution may be a molten solution of sugar alcohol. In addition, the coating solution may further include a solvent to adjust viscosity or to increase ease of manufacture. For example, the solvent may be water and/or ethanol.

For example, the coating material 130 may be formed by using a melt-blown method by spinning the coating solution. Specifically, as described above, when the coating solution including the molten solution of the sugar alcohol is discharged in the form of fine fibers by using high-pressure air or high-velocity air stream, the coating solution may be cooled in air and applied onto a surface of the packaging material 110 in the form of fibers.

According to an embodiment, the coating material 130 formed by the spinning using the melt-blown method may be in the form of fine fibers. For example, the coating material 130 may have a fineness of about 0.5 denier to about 0.8 denier or a fiber diameter of about 0.5 μm to about 1.0 μm. In other words, the coating material 130 may include fibers spun with a fineness of about 0.5 denier to about 0.8 denier or a diameter of about 0.5 μm to about 1.0 μm.

The oral pouch product 100 according to an embodiment may include the coating material 130 applied in the form of fine fibers to at least a portion of the outer surface of the packaging material 110, thereby providing a soft tactile sensation at the initial stage of use and enhancing user satisfaction.

In addition, as the sugar alcohol in the coating material 130 dissolves over the course of use, a difference in roughness between the coating material 130 and the packaging material 110 may be provided, allowing the user to more easily perceive a usage end time point. In other words, as the soft coating material 130 applied to the surface of the rough packaging material 110 dissolves, the user may perceive the usage end time point via a tactile sensation.

According to an embodiment, the packaging material 110 may include fibers having a fineness of about 2 denier to about 10 denier or a diameter of about 10 μm to about 30 μm. Due to being made of staple fibers with characteristics within the above range, a difference in roughness with respect to the coating material 130 may be increased.

The oral pouch product 100 according to an embodiment may vary the amount of the coating material 130 applied depending on the release target of the active ingredient and the solubility of the sugar alcohol with respect to saliva. For example, a small oral pouch product 100 weighing about 0.3 g to about 0.5 g may be applied such that most of the sugar alcohol may dissolve within about 20 minutes to about 30 minutes, whereas when a typical oral pouch product 100 weighing around 1 g may be applied such that most of the sugar alcohol may dissolve within about an hour. However, the disclosure is not limited thereto.

The coating material 130 may further include an edible softener. By further including the softener in the coating material 130, a tactile difference with respect to the packaging material 110 may be increased. The softener may be included in the coating solution and spun using the melt-blown method, thereby forming a coating.

The softener may be selected to be edible and suitable for a melt-blown process. For example, the softener may be one or more selected from the group consisting of citric acid, glycerin, gelatin, lecithin, alginate, pectin, guar gum, and agar.

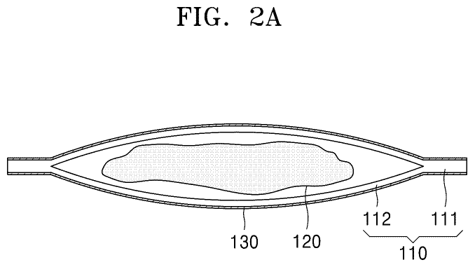

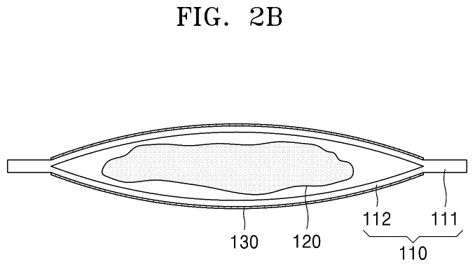

FIGS. 2A and 2B are planar cross-sectional views of an oral pouch product according to embodiments, taken along a line A-A′ of FIG. 1.

Referring to FIG. 2A, the packaging material 110 may include the sealed peripheral portion 111 and an unsealed remaining central portion 112. For example, the peripheral portion 111 of the packaging material 110 may be both end portions. For example, the central portion 112 of the packaging material 110 may refer to a portion in which the filling material 120 is enclosed.

According to an embodiment, the coating material 130 may be applied to at least a portion of the outer surface of the packaging material 110.

Referring to the embodiment of FIG. 2A, the coating material 130 may be applied over the entire surface of the packaging material 110. In other words, the coating material 130 may be applied to both an outer surface of the peripheral portion 111 and an outer surface of the central portion 112.

In an embodiment as in FIG. 2A, when the user begins using the oral pouch product 100, the sugar alcohol in the coating material 130 dissolves, allowing the user to sense a usage end time point via a tactile sensation by perceiving a difference in surface roughness between the coating material 130 and the packaging material 110.

According to another embodiment, the coating material 130 may be applied in a greater amount to an outer surface of the central portion 112 than to an outer surface of the peripheral portion 111. The coating material 130 may not be applied to the outer surface of the peripheral portion 111, or may be applied in a smaller amount to the outer surface of the peripheral portion 111 than to the outer surface of the central portion 112. Referring to FIG. 2B, the coating material 130 may be applied only to a surface of the central portion 112 of the packaging material 110.

According to the above-described embodiment, when the user beings using the oral pouch product 100, the sugar alcohol in the coating material 130 dissolves, the user may perceive a difference in surface roughness between the coating material 130 and the packaging material 110. In addition, by perceiving that a difference in surface roughness between the peripheral portion 111, where little or no coating material 130 is applied, and the central portion 112, where a relatively large amount of the coating material 130 is applied, the user may sense a usage end time point via a tactile sensation.

In addition, according to an embodiment, a method of manufacturing an oral pouch product, including the following operations, is provided.

A method of manufacturing an oral pouch product, according to an embodiment, may include: a) providing a filling material including an active ingredient; and b) sealing at least part of a peripheral portion of a packaging material and enclosing the filling material in an internal cavity of the packaging material, and may further include c) before or after operation b), applying a coating material including sugar alcohol to at least a portion of an outer surface of the packaging material.

In other words, after the filling material is enclosed in the internal cavity of the packaging material, the coating material including the sugar alcohol may be applied to a surface of the packaging material, or the filling material may be enclosed in the internal cavity of the packaging material having a surface to which the coating material including the sugar alcohol is applied.

According to an embodiment, operation c) may be performed by using a melt-blown method by spinning a coating solution including sugar alcohol onto the outer surface of the packaging material. The coating solution may be a molten solution of sugar alcohol. In addition, the coating solution may further include a solvent to adjust viscosity or to increase ease of manufacture. For example, the solvent may be water and/or ethanol.

Specifically, when the coating solution including the molten solution of the sugar alcohol is discharged in the form of fine fibers by using high-pressure air or high-velocity air stream, the coating solution may be cooled in air and applied onto a surface of the packaging material in the form of fibers.

According to another embodiment, in operation c), the coating solution may further include a softener. In operation c), as the coating solution further includes the softener, a softer coating material may be applied to the outer surface of the packaging material. The softener may be included in the coating solution and spun using the melt-blown method, thereby forming a coating. By further including the softener in the coating solution and applying the coating material, a tactile difference with respect to the packaging material may be increased.

The descriptions of the above-described embodiments are merely examples, and it will be understood by one of ordinary skill in the art that various changes and equivalents thereof may be made. Therefore, the scope of the disclosure should be defined by the appended claims, and all differences within the scope equivalent to those described in the claims will be construed as being included in the scope of protection defined by the claims.

According to an oral pouch product and a method of manufacturing the same, according to embodiments, a usage end time may be easily and conveniently perceived without having to check whether a recommended usage time has elapsed.

The oral pouch product according to the embodiments allows a user to perceive a usage time via texture felt on a surface of the pouch product during use, making it easier to stop usage before experiencing a foreign-body sensation in the throat, gastrointestinal discomfort, or nausea.

Effects of the present disclosure are not limited to the above effects, and effects that are not mentioned could be clearly understood by one of ordinary skill in the art from the present specification and the attached drawings.

What is claimed is:

1. An oral pouch product comprising:
a packaging material having at least part of a peripheral portion sealed to define a cavity therein;
a filling material enclosed in the cavity and comprising an active ingredient; and
a coating material applied to at least a portion of an outer surface of the packaging material and comprising sugar alcohol.

2. The oral pouch product of claim 1, wherein the coating material is formed by using a melt-blown method by spinning a coating solution including sugar alcohol onto the outer surface of the packaging material.

3. The oral pouch product of claim 2, wherein the coating material comprises fibers having a fineness of about 0.5 denier to about 0.8 denier or a diameter of about 0.5 μm to about 1.0 μm.

4. The oral pouch product of claim 1, wherein the sugar alcohol is one or more selected from the group consisting of isomaltol, erythritol, xylitol, sorbitol, maltitol, and mannitol.

5. The oral pouch product of claim 1, wherein the coating material further comprises a softener.

6. The oral pouch product of claim 5, wherein the softener is one or more selected from the group consisting of citric acid, glycerin, gelatin, lecithin, alginate, pectin, guar gum, and agar.

7. The oral pouch product of claim 1, wherein the packaging material comprises the sealed peripheral portion and a remaining central portion, and
the coating material is applied in a greater amount to an outer surface of the central portion than to an outer surface of the peripheral portion.

8. The oral pouch product of claim 7, wherein the coating material is not applied to the peripheral portion, and the coating material is applied to the central portion.

9. The oral pouch product of claim 1, wherein the packaging material comprises fibers having a fineness of about 2 denier to about 10 denier or a diameter of about 10 μm to about 30 μm.

10. The oral pouch product of claim 1, wherein the active ingredient comprises nicotine.

11. The oral pouch product of claim 10, wherein the nicotine is one or more selected from the group consisting of nicotine hydrochloride, nicotine dihydrochloride, nicotine monotartrate, nicotine bitartartrate, nicotine bitartartrate dihydrate, nicotine sulfate, nicotine zinc chloride monohydrate, nicotine salicylate, nicotine benzoate, and nicotine polacrilex.

12. A method of manufacturing an oral pouch product, the method comprising:
a) providing a filling material including an active ingredient; and
b) sealing at least part of a peripheral portion of a packaging material and enclosing the filling material in an internal cavity of the packaging material,
wherein the method further comprises c) before or after operation c), applying a coating material including sugar alcohol to at least a portion of an outer surface of the packaging material.

13. The method of claim 12, wherein operation c) is performed by using a melt-blown method by spinning a coating solution including sugar alcohol onto the outer surface of the packaging material.

14. The method of claim 13, wherein, in operation c), the coating solution further comprises a softener.
